(19) United States
(12) Patent Application Publication
(10) Pub. No.: US 2026/0002205 A1
(43) Pub. Date: Jan. 1, 2026

(54) METHOD OF IDENTIFYING ALLOSTERIC BIOSENSOR PROTEINS WITH NEW SPECIFICITIES

(71) Applicant: Wisconsin Alumni Research Foundation, Madison, WI (US)

(72) Inventors: Srivatsan Raman, Middleton, WI (US); Kyle Nishikawa, Middleton, WI (US); Nathan James Novy, Madison, WI (US)

(73) Assignee: Wisconsin Alumni Research Foundation, Madison, WI (US)

(21) Appl. No.: 18/653,004

(22) Filed: May 2, 2024

Related U.S. Application Data
(60) Provisional application No. 63/499,615, filed on May 2, 2023.

(51) Int. Cl. C12Q 1/6869; C12N 15/10; C12Q 1/6806; C12Q 1/6897; C40B 40/02

(52) U.S. Cl. CPC C12Q 1/6869; C12N 15/1003; C12N 15/1034; C12Q 1/6806; C12Q 1/6897; C40B 40/02

(57) ABSTRACT

Described herein is a method of selecting allosteric biosensor proteins which bind a target ligand. The method includes providing a library of replicating plasmids each including an expression construct and a reporter, wherein each expression construct includes a gene encoding the allosteric protein variant and the reporter, wherein the reporter includes a barcode sequence for identification of the allosteric protein variant or allosteric domain variant. The method further includes mapping the variants in the library to the barcode sequence or sequences associated with the variant and assigning variant-barcode pairs, growing a population of cells transfected with the library of replicating plasmids in the presence of the target ligand and isolating target ligand total RNA and target ligand library plasmids; performing next generation sequencing to determine a quantity of each barcode in the target ligand total RNA, determining a fold enrichment for each allosteric protein variant or allosteric domain variant in the target ligand total RNA, and selecting a subpopulation of variants with the highest fold enrichment as the selected allosteric biosensors.

### CROSS-REFERENCE TO RELATED APPLICATIONS

This application claims priority to U.S. Provisional Application 63/499,615 filed on May 2, 2023, which is incorporated herein by reference in its entirety.

### STATEMENT REGARDING FEDERALLY SPONSORED RESEARCH & DEVELOPMENT

This invention was made with government support under W911NF-20-C-0005 awarded by the ARMY/ARO. The government has certain rights in the invention.

### BACKGROUND

Allosteric transcription factors (aTFs) control gene expression in response to changes in the environment. Prokaryotic transcription factors such as LacI or TetR have the capacity to detect small molecules and have a simple mechanism of gene expression control, making these proteins logical candidates for biosensing applications. In the absence of the small molecule inducer, the transcription factor remains bound to the operator sequence in the promoter of controlled genes, physically preventing RNA polymerase from interacting with the promoter. aTF binding to the inducer causes an allosteric change that decreases affinity for the operator sequence, allowing downstream gene expression.

A major limitation of implementing transcription factor biosensors is the narrow range of molecules that can be bound by characterized transcription factors. Designing aTFs and other allosteric biosensor proteins for novel ligand affinity has two major challenges: mutating the ligand binding pocket for affinity for the target molecule and maintaining allosteric function. The methods described herein overcome these shortcomings.

### BRIEF SUMMARY

In an aspect, a method of selecting allosteric biosensor proteins which bind a target ligand comprises
providing a library of replicating plasmids each plasmid comprising an expression construct and primer binding sites for next generation sequencing of at least a portion of an allosteric protein variant or an allosteric domain variant and a reporter, wherein each expression construct comprises a gene encoding the allosteric protein variant or the allosteric domain variant which is operably linked to a first promoter for expression of the allosteric protein variant or allosteric domain variant, and functionally linked to the gene, is the reporter comprising a barcode sequence for identification of the allosteric protein variant or allosteric domain variant, wherein the reporter is operably linked to a second promoter,
wherein, when the target ligand binds expressed allosteric protein variant or allosteric domain variant, expression of the reporter from the second promoter is activated, and when the target ligand does not bind expressed allosteric protein variant or allosteric domain variant, expression of the reporter from the second promoter is inactivated, or wherein, when the target ligand binds expressed allosteric protein variant or allosteric domain variant, expression of the reporter from the second promoter is inactivated, and when the target ligand does not bind expressed allosteric protein variant or allosteric domain variant, expression of the reporter from the second promoter is activated;
mapping each allosteric protein variant or allosteric domain variant in the library to the barcode sequence or sequences associated with the allosteric protein variant or allosteric domain variant and assigning variant-barcode pairs;
growing a population of cells transfected with the library of replicating plasmids in the presence of the target ligand and isolating target ligand total RNA and target ligand library plasmids;
performing next generation sequencing to determine a quantity of each barcode in the target ligand total RNA; and either
(i) from the quantity of each barcode and the assigned variant-barcode pairs determining a fold enrichment for each allosteric protein variant or allosteric domain variant in the target ligand total RNA, wherein the fold enrichment for each allosteric protein variant or allosteric domain variant in the target ligand total RNA is normalized by the target ligand library plasmid; and selecting a subpopulation of variants with the highest fold enrichment as the selected allosteric biosensors, or
(ii) treating each barcode for a specific variant as a technical replicate, applying estimation with restricted maximum likelihood (RML) to combine the technical replicates, performing a second round of RML to merge biological replicates; and selecting a subpopulation of merged biological replicates as the selected allosteric biosensors.

### BRIEF DESCRIPTION OF THE DRAWINGS

FIG. 1 illustrates the principle of the method described herein wherein binding of a target ligand by an allosteric protein variant or allosteric domain variant can be determined by transcript abundance. On the left, in the absence of ligand binding, little or no transcription occurs. On the right, in the presence of bound ligand, transcription is activated. As described herein, an RNA barcode reporter is used to determine transcript abundance.

FIGS. 2A-H show validating RNA-Seq on a 16-member library. (2A) The space of transcription factors and small molecules that can be sensed with different approaches. Black points represent specific ligand: aTF pairs. The small dark circle represents the extent to which methods can currently expand aTF: ligand affinity. The light circles represents the extent to which new methodologies must increase aTF: ligand pairs. (2B) Construct design pairs aTF variants to randomized barcodes. (2C) A separate construct is used to pair barcodes to aTF variants such that RNA-Seq of barcodes can be translated to aTF function. (2D) Methodology to harvest plasmids and RNA from E. coli transformed with aTF libraries. This approach can easily be scaled across multiple ligands and multiple libraries. (2E) The number of barcodes per variant identified using next-generation sequencing of the construct in (2C). Each variant is identified by a separate binary string. (2F) Box plots of fold enrichment for each variant via RNA-Seq. The box represents the interquartile range. Whiskers extend to 1.5 times the IQR. Fliers denote points that lie outside the whiskers. Variants are represented by binary strings. (2G) Correlation of qRT-PCR data and fold enrichment from RNA-Seq. qRT-PCR fold enrichment was measured via biological replicates of clonal strains of 8 of the 16 variants (see methods). The RNA-Seq fold enrichment value was calculated by summing the counts of all barcodes associated with a particular variant (see methods). The R2 for this dataset is 0.83. (2H) Bootstrap correlation of qRT-PCR fold enrichment to RNA-Seq data for 8 of the 16 variants. Groups of 10, 25, 50, 100, or 500 barcodes were sampled for each variant across 500 cycles. The resulting correlation for each cycle is plotted.

FIGS. 3A-C show identifying novel sensors in the agnostic library. (3A) The top 40 best variants from each ligand were selected for a fluorescence-based screen (individual points). The violin plot shows the fold enrichment calculated by RNA-Seq for all 17,365 variants for each ligand. The circle inside the violin plot denotes the median of fold enrichment for each ligand. The thick grey line inside the violin plot represents the IQR and the thin grey line extends to 1.5 times the IQR. (3B) Fluorescence screening workflow that incorporates a construct with sfGFP. A single repressed sort and an induced sort were sequenced (see methods). Fold change (FC) was calculated as the ratio of percent change in the population with and without ligand. (3C) Fold change for each ligand across the 251 best performing variants.

FIGS. 4A-E show elucidating ligand-specific sequence preferences from RNA-Seq. (4A) RNA-Seq fold enrichment data for 3,3135 variants across nine ligands. Ligands and variants have been clustered via the UPGMA algorithm with a correlation distance metric and a target of 12 clusters (see methods). The different clusters are denoted by the bars on the right of the heatmap. aTF function is shown as the log 2 (fold enrichment) normalized to wildtype. (4B) Structure of TtgR with tolerated mutations at each position (PDB ID: 7K1C). The wildtype residue is highlighted at each position as purple sticks. Resveratrol, a natural ligand of TtgR, is shown. The tolerated mutations at each position are shown with the darker background while the wildtype identity is shown in white. (4C) Sequence relative positional entropy of the clustered data for all tolerated positions. In this plot, a higher relative entropy indicates a changed amino acid distribution after clustering. (4D) Heatmap of the fold change in amino acid abundance across allowed positions for clusters 7, 11, and 12. The fold change of frequency is the log 2 of the ratio of amino acid frequency after clustering to the frequency before clustering. (4E) Heatmap of the fold change in amino acid abundance across allowed positions for the top 40 variants for each ligand. Fold change is calculated identical to (3D).

FIGS. 5A-C show DMS of TtgR against endoxifen and tamoxifen highlights functional hotspots. (5A) Heatmap of DMS library performance when exposed to endoxifen. White squares are positions and mutations that did not pass the CV filter (see methods). The remaining positions are coded by the fold enrichment of the mutant normalized to wildtype. The diagram on the right of the heatmap shows the location of alpha helices (rectangles) and disordered loops (lines). The DNA binding domain helices are 1-76 while the ligand binding domain helices are 81-206. (5B) Heatmap of DMS library performance when exposed to tamoxifen. Coding is identical to (A). (5C) Functional hotspots of TtgR. Positions defined as hotspots are shown (PDB ID: 7K1C). Resveratrol, a native ligand of TtgR, is shown.

FIGS. 6A-I shows structures of (6A) tamoxifen, (6B) endoxifen, (6C) 4-hydroxytamoxifen, (6D)N-desmethyltamoxifen, (6E) ellagic acid, (6F) quinine, (6G) naltrexone, (6H) naringenin, and (6I) resveratrol. Hydrogens are not shown.

FIG. 7 shows shared sequences in top performing variants. The top 40 variants for each ligand were selected and listed (x-axis). Variants are marked if they are within the top 40 for a particular ligand. There are 251 unique variants in the set of top performers across all ligands.

FIG. 8 shows a heatmap showing fold induction of wild-type TtgR and variants that were selected as top 3 for each ligand based on sorting.

FIGS. 9A-D show improvements to the SensorSeq methodology. FIG. 9A shows the correlation between biological replicates for each condition using old and new approaches for fitness scores. FIG. 9B shows the correlation between fitness scores determined by SortSeq fluorescence validation of results to the SensorSeq fitness cores calculated using new and old methods. FIG. 9C shows scatter plots of the confidence scope for each measurement and the fold induction SensorSeq score determined with the improved fitness calculation method. The gray bar is scaled by the product of the confidence score and the Sensor Seq score. Darker points have a higher likelihood of being highly sensitive biosensors. FIG. 9D shows the correlation between low and high volume SensorSeq assays for the native ligand naringenin.

The above-described and other features will be appreciated and understood by those skilled in the art from the following detailed description, drawings, and appended claims.

### DETAILED DESCRIPTION

Engineering novel ligand affinity into allosteric transcription factors and other proteins has enormous importance in biotechnology, where these proteins can serve as natural biosensors. Efforts to engineer these proteins has been limited due to extensive long-range interactions that create the allosteric response. In the absence of prior knowledge of these interactions, novel function must be found through many empirical measurements of function. Described herein is a novel computational design and high-throughput screening workflow that incorporates ligand-agnostic variants with RNA-Seq to engineer new biosensors. This approach generated variants with affinity to nine nonnative ligands. Sequence analysis of high performing variants revealed distinct sequence profiles for different ligand specificities. The screening workflow was also applied to characterize functional hotspots in a DMS library, revealing important locations at the interface between the ligand binding domain and the DNA binding domain. This workflow can be applied to screen function in any protein whose function is a measurable change in transcription.

More specifically, described herein is a ligand-agnostic computational design approach coupled with an RNA-Seq workflow for quantitative analysis of transcription factor function referred to as SensorSeq. In the examples, the evolutionary history of TtgR was leveraged to create a library of phylogenetically derived, computationally stable amino acid substitutions at key locations in the binding pocket. A library of TtgR variants was screened against nine different ligands for functional aTFs to show that RNA-Seq is also applicable in mutational scanning libraries by screening a TtgR deep mutational scanning (DMS) library against endoxifen and tamoxifen. Groups of variants change gene expression in response to each ligand and top performing variants were validated in a fluorescence-based assay. Furthermore, the variant library contains unique patterns of ligand specificity across all tested ligands, which are reflected by amino acid preferences at important positions. Finally, the DMS screen is able to identify allosterically important regions connecting the DNA binding domain and ligand binding pocket. This work establishes a novel approach to create new biosensors, e.g., transcription factor biosensors that is also pertinent to basic science applications and can be applied to any protein whose functional readout can be quantified via transcription.

As used herein, an allosteric biosensor protein is a protein with an activity that is altered when it binds to a target ligand. When the allosteric protein binds a target ligand it undergoes a conformational change resulting in an increase or decrease in activity. A well-known example of an allosteric protein is an allosteric transcription factor as illustrated in FIG. 1. The methods described herein are not, however, limited to transcription factors, but rather are applicable to any protein wherein binding to a target ligand can be measured by a transcriptional readout. For example, the methods can be applied to any protein whose activity can be relayed through a signaling cascade to a transcriptional reporter.

In an aspect, a method of selecting allosteric biosensor proteins which bind a target ligand comprises first providing a library of replicating plasmids each plasmid comprising an expression construct and primer binding sites for next generation sequencing of at least a portion of an allosteric protein variant or an allosteric domain variant and a reporter.

The library could be generated from any source such as computational-guided design, ML generative models, random mutagenesis, DNA shuffling, and the like. The downstream workflow described herein is agnostic to how the library is generated.

The expression construct comprises a gene encoding the allosteric protein variant or the allosteric domain variant which is operably linked to a first promoter for expression of the allosteric protein variant or allosteric domain variant. Functionally linked to the gene is a reporter comprising a barcode sequence for identification of the allosteric protein variant or allosteric domain variant, wherein the reporter is operably linked to a second promoter.

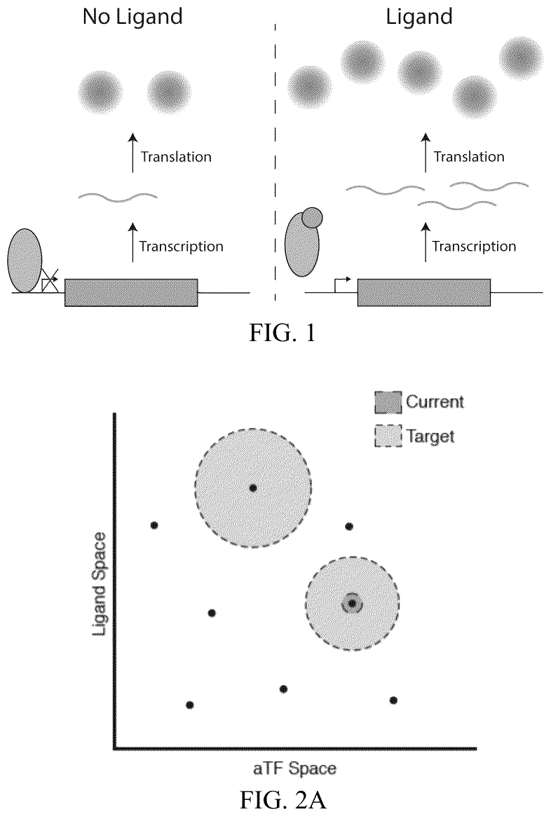

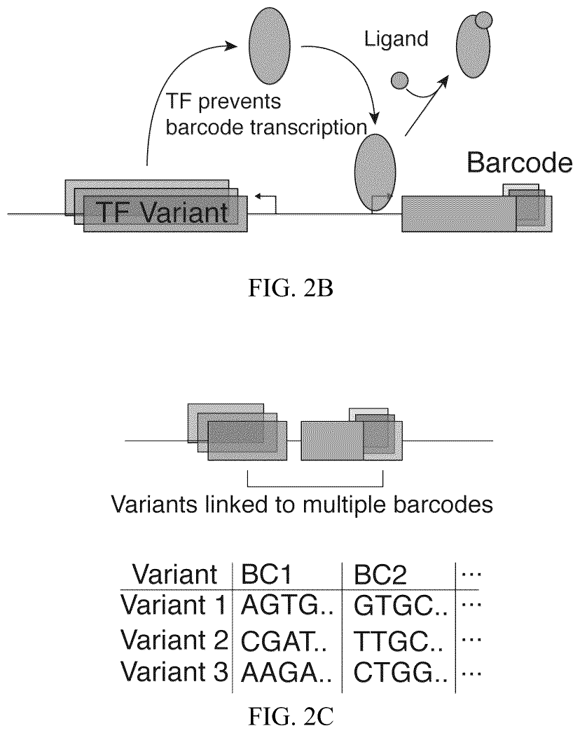

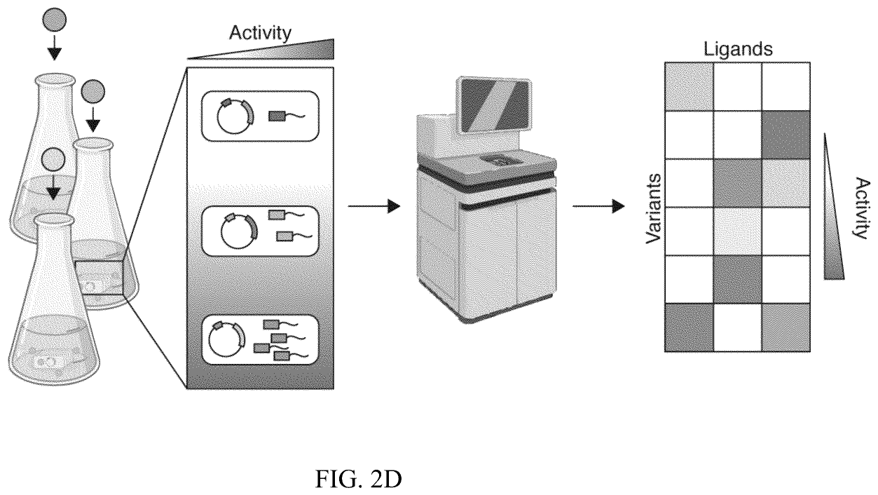

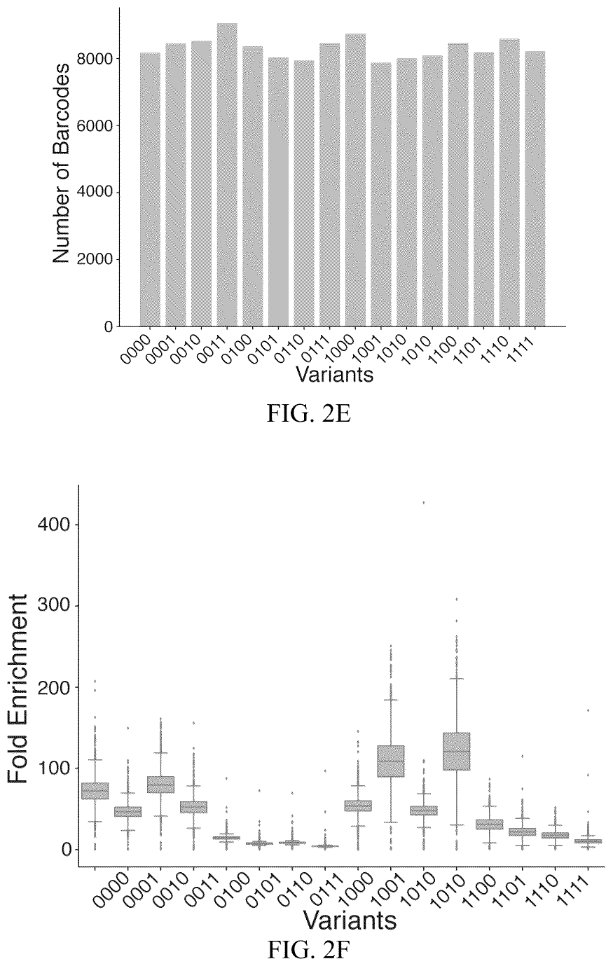

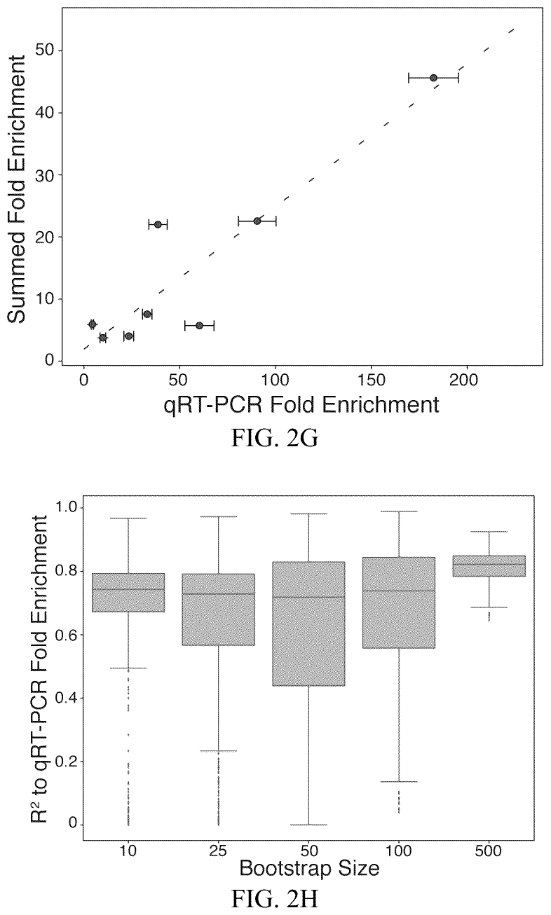

As illustrated in FIG. 2B, an embodiment of the expression construct comprises the gene encoding the allosteric protein variant or the allosteric domain variant (exemplified as a TF variant) operably linked to a first promoter for expression of the allosteric protein variant or allosteric domain variant, represented by the arrow pointing to the left. Functionally linked to the TF variant gene is a reporter comprising a barcode sequence for identification of the allosteric protein variant or allosteric domain variant, wherein the reporter is operably linked to a second promoter, represented by an arrow pointing to the right. While transcription of the TF variant gene and the reporter occur in opposite directions in this example, this is not necessary. Multiple boxes for the TF variant and the barcode represent the library members.

The allosteric protein variant or allosteric domain variant can have any number of variable amino acids to provide the target variability in the library.

In a first aspect, when the target ligand binds expressed allosteric protein variant or allosteric domain variant, expression of the reporter from the second promoter is activated, and when the target ligand does not bind expressed allosteric protein variant or allosteric domain variant, expression of the reporter from the second promoter is inactivated or not activated. This is illustrated in FIG. 1. In this case, the target ligand is an allosteric activator. In a second aspect, when the target ligand binds expressed allosteric protein variant or allosteric domain variant, expression of the reporter from the second promoter is inactivated or not activated, and when the target ligand does not bind expressed allosteric protein variant or allosteric domain variant, expression of the reporter from the second promoter is activated. In this case the target molecule is an allosteric inhibitor.

In an aspect, the allosteric protein or allosteric domain comprises a DNA binding domain that, in the presence of the target ligand, either inactivates or activates expression of the reporter from the second promoter. Exemplary proteins comprising a DNA binding domain include allosteric transcription factors such as TetR, LacI, TtgR, MphR, AraC, LysR. By way of example, TtgR belongs to the TetR family of proteins, which are characterized by a structurally conserved DNA binding domain and variable ligand binding domain.

In another aspect, the allosteric protein or allosteric domain comprises a multi-component system such as a two-component system in which the allosteric protein or allosteric domain are functionally linked to a DNA binding domain of a different protein. In this aspect, the allosteric protein or allosteric domain associates, directly or indirectly, with a DNA binding domain that, in the presence of the target ligand, either inactivates or activates expression of the reporter from the second promoter. Upon target ligand binding, the target ligand binding domain relays the signal through one or more intermediary proteins, resulting in transcription regulation at a defined locus. Exemplary allosteric protein or allosteric domain/DNA binding protein pairs include PhoP/PhoQ, EnvZ/OmpR, KdpE/KdpD, ComA/ComP, and nuclear hormone receptors. Additional multi-component systems can be identified using two hybrid screens, for example. Table 1 provides exemplary transcription factors.

TABLE 1
EXEMPLARY TRANSCRIPTION FACTORS

| | Transcription | Example transcription |
|---|---|---|
| System | factor family | factors |
| Prokaryotes-One | LTTR | LysR, AtzR, AlsR, ArgP, |
| component | | BenM, CidR, CynR, HvrB, |
| | | ToxR, OccR, NodD, NocR, |
| | | NahR IlvY, MetR, MdcR, |
| | | CysB |
| | AraC/XylS | AraC, XylS, RhaR, RhaS, |
| | | UreR, Rob, Rns, MelR |
| | DeoR | DeoR, GlpR, GutR, FucR, |
| | | UlaR, LacR, FruR, IolR, AccR |
| | DtxR | DtxR, SloR, MtsR, MntR |
| | Fur | Fur, Zur, Mur, Nur |
| | GntR | GntR, FadR, HutC, MocR, |
| | | YtrA, AraR, DevA, PlmA |
| | LuxR | LuxR, AhyR, QscR, HapR |
| | Lrp/AsnC | Lrp, AsnC, LrpA, LrpC |
| | Crp/Fnr | Crp, FnR, Vfr, |
| | IclR | IclR, RexZ, SsfR, |
| | MerR | MerR, BltR, BmrR, Mta, |
| | | CueR, ZntR, PbrR |
| | MarR | MarR, OhrR, SlyA, MosR, |
| | | RovA, MepR |
| | LacI/GalR | LacI, CcpA, GalR, PurR, |
| | | CytR, CRA |
| | Amino acid | ArgR, Trp |
| | metabolism | |
| | TetR | TetR, AcuR, AguR, BepR, |
| | | CmeR, ComR, CymR, DesT, |
| | | EthR, FabR, FrrA, HdnoR, |
| | | HrtR, LanK, LrfR, LmrA, |
| | | NalC, MphR, PaaR, Pip, |
| | | QacR, RolR, SimR, SmeT, |
| | | TtgR, VarR, VceR |
| Prokaryotes- two | | PhoP/PhoQ, EnvZ/OmpR, |
| component | | KdpE/KdpD, ComA/ComP |
| Eukaryotes | Nuclear receptor | NR3C1, NR3A1, NR3C2 |

“Operably linked” means that the nucleotide sequence of interest is linked to the regulatory sequence(s) in a manner that allows for expression of a gene. “Functionally linked” means that the product of one component is able to interact with another component or the product of another component. For example, a gene encoding the allosteric protein variant or the allosteric domain variant is functionally linked to the reporter comprising the barcode so that the expressed allosteric protein variant or allosteric domain variant protein interacts with the second promoter to regulate expression of the barcode.

In an aspect, the first promoter for expression of the allosteric protein variant or allosteric domain variant is a constitutively active promoter such the apFAB61 promoter with BBa_J61132 ribosome binding site. Additional constitutively active promoters can be found in the Registry of Standard Biological Parts.

In an aspect, the second promoter for expression of the reporter is an inducible promoter, which depends upon the DNA binding domain regulated by the allosteric protein or allosteric domain.

In an aspect, the barcodes have length of 16 to 24 nucleotides, although shorter and longer barcodes may be employed.

In an aspect, the library comprises 5,000 to 20,000 allosteric protein variants or allosteric domain variants.

The method also includes mapping each allosteric protein variant or allosteric domain variant in the library to the barcode sequence or sequences associated with the variant and assigning variant-barcode pairs. Preferably, each allosteric protein variant or allosteric domain variant in the library is associated with 10 to 100 barcodes.

Mapping can comprise direct sequencing of the expression constructs. For example, PacBio or Oxford nanopore long read sequencing can be used to assign the variant-barcode pairs. Alternatively, mapping can comprise removing a constant region of the expression constructs of the library of plasmids between the variable region of the allosteric protein variant or allosteric domain variant and the barcodes, and ligating the variable region to the barcode sequence, and performing high throughput sequencing such as Illumina® sequencing to assign the variant-barcode pairs. This method is illustrated in FIG. 2C.

As illustrated in FIG. 2D, the method next includes growing a population of cells transfected with the library of replicating plasmids in the presence of the target ligand and isolating target ligand total RNA and target ligand library plasmids. As used herein, the terms “transformation” and “transfection” are intended to refer to a variety of art-recognized techniques for introducing foreign nucleic acid into a host cell. The target ligand total RNA is used to quantify the amount of each barcode expressed, and the target ligand library plasmids are used to normalize the quantity of each barcode expressed and account for library skew.

Exemplary cells include gram positive bacteria such as Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae, and the like. Exemplary cells also include gram negative bacteria such as Afipia felis, Bacteroides, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis, and the like.

As illustrated in FIGS. 2E and 2F, in a first aspect (i), from the quantity of each barcode and the assigned variant-barcode pairs, a fold enrichment for each allosteric protein variant or allosteric domain variant in the target ligand total RNA is determined. The fold enrichment for each allosteric protein variant or allosteric domain variant in the target ligand total RNA is normalized by the target ligand library plasmid. For example, the RNA counts in the +ligand state are normalized to the DNA counts in the +ligand state first and then divided by the same ratio for the −ligand state. See Eq.4 in the Examples.

A subpopulation of variants with the highest fold enrichment are identified as the selected allosteric biosensors. In an aspect, selecting the subpopulation of variants with the highest fold enrichment as the allosteric biosensors comprises selecting a desired number of top variants, such as 20 to 100 variants. The number of variants selected will depend on the size of the library and other factors.

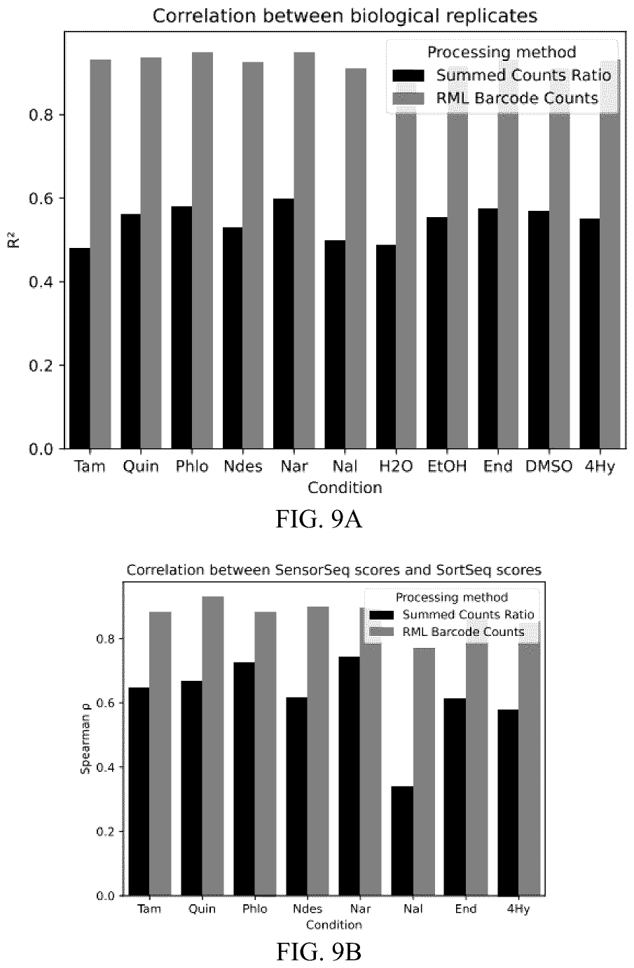

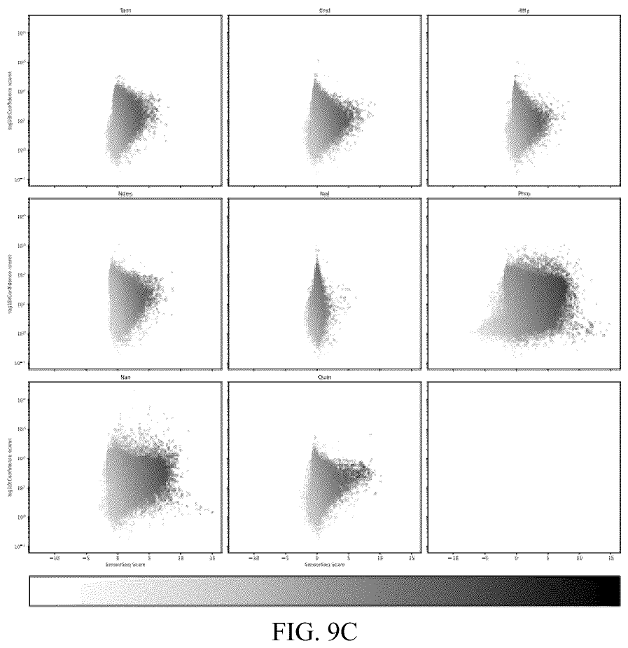

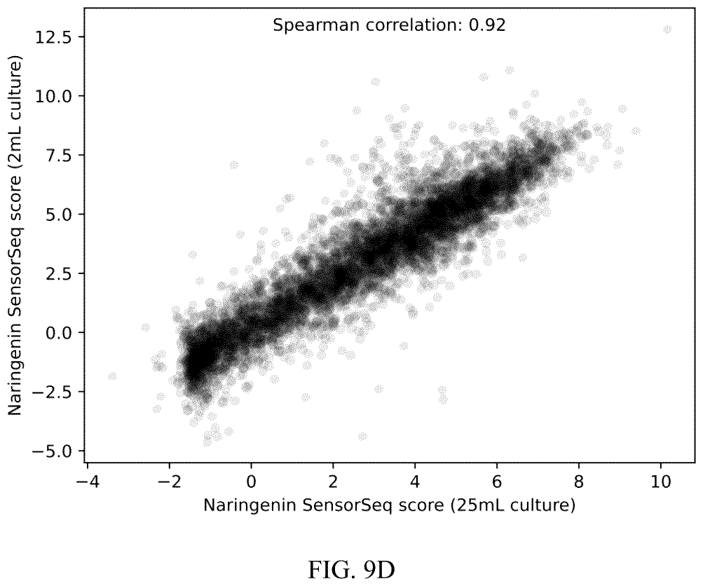

In a second aspect (ii), as illustrated in FIG. 9, the method can include treating each barcode for a specific variant as a technical replicate, applying estimation with restricted maximum likelihood (RML) to combine the technical replicates, performing a second round of RML to merge biological replicates; and selecting a subpopulation of merged biological replicates as the selected allosteric biosensors. Optionally, the method further comprises determining a functional score for each of the selected allosteric biosensors using confidence scoring.

In an aspect, selecting allosteric biosensor proteins is done in a high throughput assay.

Optionally, in addition to the barcode, the reporter can also comprise a coding sequence for a detectable marker protein also operably linked to the second promoter. The coding sequence for a detectable marker protein is represented by a green box in FIG. 2B. Advantageously, the detectable marker protein can provide a readout for validation of the allosteric biosensor proteins selected in the method, although it is not necessary for the initial screen for allosteric biosensor proteins with the highest enrichment. Exemplary detectable marker proteins comprise a green fluorescent protein (GFP), a blue fluorescent protein (BFP), a yellow fluorescent protein (YFP), a violet-excitable green fluorescent variant protein, enhanced green fluorescent protein or EGFP, a red fluorescent protein, and an orange fluorescent protein.

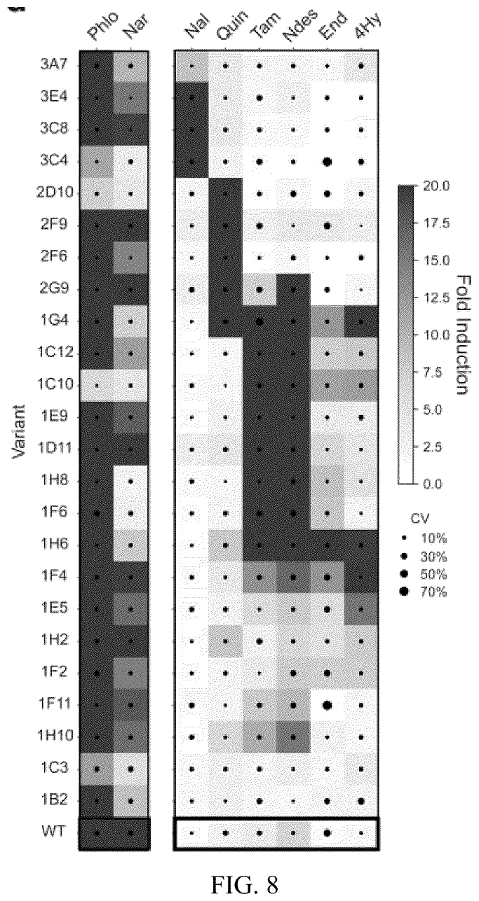

In an aspect, as shown in FIG. 8, the method optionally comprises validating the subpopulation selected allosteric biosensors by determining expression of the detectable marker protein in the presence and absence of the target ligand.

Exemplary target ligands include a biological molecule, an environmental molecule, a drug, a metal ion, a carcinogenic molecule, a food contaminant, an environmental contaminant, human wellness indicators such as hormones, cellular metabolites, signaling molecules inside and outside the cell, and the like.

A method of detecting a target ligand in a sample comprises contacting the sample with the allosteric biosensor protein selected according to the methods described herein, and detecting the presence or absence of the target ligand in the sample. Exemplary samples include biological samples (blood, serum, plasma, urine, saliva, cerebrospinal fluid or amniotic fluid, a tissue sample such as a tissue or organ biopsy or may be a cellular sample such as a sample comprising red blood cells, lymphocytes, tumor cells or skin cells), environmental samples, food samples, industrial samples, and the like.

Also included is a device comprising the allosteric biosensor protein selected by the methods described herein. A device may also include a substrate, cell or chamber comprising the allosteric biosensor protein. Exemplary substrates include a plastic, a cellulose product such as paper, polymer, metal, noble metal, semi-conductor, or quantum dot. The device also includes a means for identifying the presence or absence of the target ligand in the sample based on the interaction of the target ligand with the allosteric biosensor protein. The means may be a colorimetric or fluorescent signal.

The device may be used in high throughput screening, single cell analysis, online monitoring, evolution, or dynamic pathway evolution, cell-free biosensing, whole-cell biosensing, control of cellular functions, inducible promoters for gene expression control, and the like.

The invention is further illustrated by the following non-limiting examples.

### EXAMPLES

### Methods

Plasmid creation: Amplicons were generated using Kapa HiFi™ (Roche) PCR kits following the manufacturer protocol. Amplicons were treated with 15U of Dpn1 (NEB) for 2.5 hours at 37° C. followed by 20 minutes at 80° C. PCR amplicons were then purified using E.Z.N.A.® Cycle Pure kits (Omega BioTek). Isothermal assembly followed Gibson Assembly protocols (NEB), but contained 100 mM Tris-HCl pH 7.5, 20 mM MgCl₂, 0.2 mM dATP, 0.2 mM dCTP, 0.2 mM dGTP, 10 mM dTT, 5% PEG-8000, 1 mM NAD⁺, 4 U/ml T5 exonuclease, 4 U/μl Taq DNA ligase, and 25 U/ml Phusion® polymerase. Isothermal assembly reactions were diluted 10× in dH₂O prior to transformation. DH10B (NEB) electrocompetent cells were transformed with 2 μL of diluted isothermal assembly reaction. Transformants were recovered in 700 μL SOC medium (2% tryptone, 0.5% yeast extract, 10 mM NaCl, 2.5 mM KCl, 10 mM MgCl₂, 10 mM MgSO₄, and 20 mM glucose) for 1 hour at 37° C. Dilutions were plated on LB-kanamycin (50 μg/mL) plates and incubated at 37° C. overnight. Colony PCR was performed using Kapa2G Robust™ (Roche) using a single colony diluted in 100 μL of dH₂O. Plasmid purifications were performed using the ZR Plasmid Miniprep™ Classic kit (Zymo).

Library creation: Plasmid libraries were generated using NEB® Golden Gate Assembly Kits (NEB, BsaI-HFv2). The reactions underwent a cycling protocol of 30 alternating 5-minute 37° C. and 16° C. cycles followed by a final 60° C. 5-minute hold. The reactions were dialyzed against dH₂O on semi-permeable membranes (Millipore) for 1 hour at room temperature. DH10B (NEB) cells were transformed with 3 μL of dialyzed reaction via electroporation. Transformants were recovered in 1 mL of SOC and then diluted 2×, 5×, and 10× with fresh SOC. Each dilution recovered for 1 hour shaking at 37° C. 4 mL of LB-kanamycin (50 μg/mL) was added to each dilution after recovery and 50× and 500× dilutions were plated of each recovered dilution to calculate transformation efficiency. The remaining transformants were grown for 6 hours shaking at 225 rpm. A frozen stock was made in 25% glycerol and stored at −80 for each dilution. Fresh cultures were created by diluting each 6-hour growth 50× into fresh LB-kanamycin. These were grown overnight and plasmids were harvested via ZR Plasmid Miniprep™ Classic kit (Zymo).

RNA purification: Cells were struck out on an LB-kanamycin plate and grown overnight at 37° C. Three colonies were inoculated into LB-kanamycin for overnight growth. The overnight cultures were diluted 50× into fresh LB-kanamycin containing either ligand or solvent. The cultures were grown at 37° C. shaking at 250 rpm in an Innova 4230 (New Brunswick Scientific). At the targeted OD600, cultures were placed on ice for 10 minutes. 5*10⁸ cells were harvested by centrifugation at 5,500 g based on the OD600 and the assumption that 1.0 OD600 cultures have 8*10⁸ cells/mL. The pelleted cells were decanted and stored at −80° C. This process was repeated in biological triplicate for each target OD600 with new colonies.

RNA was purified from cell pellets via TRIzol® reagent (Invitrogen). 1 mL of TRIzol® reagent (Invitrogen) was added to each cell pellet and vortexed briefly. The samples were incubated at room temperature for 5 minutes. 200 μL of chloroform (Sigma Aldrich) was added to each sample. The samples were incubated at room temperature for 2 minutes and were centrifuged at 12,000 g for 15 minutes at 4° C. 300 μL of the aqueous phase was transferred to a clean 2 mL centrifuge tube and placed on ice. RNA was purified from the aqueous phase using the RNA Clean & Concentrator™ 5 kit (Zymo) and eluted in 15 μL Ultrapure RNase-free dH₂O (Invitrogen). The purified RNA was digested using 4U DnaseI (NEB) in a 50 μL reaction incubated at 37° C. for 30 minutes. The digestion reactions were purified using the RNA Clean & Concentrator™ 5 kit (Zymo) and eluted in 15 μL Ultrapure RNase-free dH2O (Invitrogen). Concentrations were measured using a Nanodrop™ instrument (Thermo Fisher).

qRT-PCR quantification of transcript abundance: The abundance of the sfGFP and rrsA transcripts were measured via qRT-PCR. Each biological triplicate RNA was run in technical triplicate in a MicroAmp™ Fast Optical 96-well plate (Life Technologies). Ing of RNA was added to Luna® Universal One-Step qRT-PCR mix (NEB) containing 4 μmol of each primer on ice. The standard cycling protocol was used according to the manufacturer's suggestion. Each sample consisted of a set of reactions containing sfGFP specific primers and another set containing rrsA-specific primers. The reactions were run on a CFX Connect™ Real Time PCR Detection System (BioRad). Fold enrichment was calculated using equations (1) and (2). The error was propagated from the technical replicates and biological replicates using (3).

$$fold enrichment = {2}^{- ΔΔ {C}_{t}} \qquad ( 1 )$$

$$ΔΔ {C}_{t} = {( {C}_{t GFP} - {C}_{t rrsA} )}_{+ Ligand} - {( {C}_{t GFP} - {C}_{t rrsA} )}_{- Ligand} \qquad ( 2 )$$

$$error = \sqrt{\sum ( {σ}_{i} )} \qquad ( 3 )$$

Short barcode oligo synthesis: Pre-defined or random barcodes were synthesized as a short primer (IDT). These barcode primers were combined separately with another constant primer to create short double-stranded fragments containing the barcode flanked by BsaI cut sites in a single cycle of PCR using Kapa HiFi™ (Roche). 1 μL of this reaction was added into a second Kapa HiFi™ (Roche) reaction with additional primers to increase the length of the amplicon over 18 cycles. The resulting amplicon was purified using the DNA Clean & Concentrator™ 5 kit (Zymo).

Barcode-variant mapping via next-generation sequencing: Two primer groups were used to add Illumina® sequencing regions to the barcode-spacer-variant region of the mapping plasmid libraries. Each primer group consisted of three primers with different numbers of Ns (ON, 3N, or 6N) to increase positional base diversity during runs. The adapter primers had complementarity to the plasmid and contained Illumina® sequencing primer binding regions. Stem primers had the i7 and i5 indices and the adapter sequence to anneal to the sequencing flow cell. The adapter regions were added using Ing of template, 0.6 μL of 10 μM primers, and Kapa HiFi™ mix (Roche) for 14 cycles. These reactions were purified using the DNA Clean & Concentrator™ 5 kit (Zymo). The stem primers were used in a second PCR reaction using 4 μL of the first reaction for 10 cycles.

Sample preparation for sequencing: For MiSeq-based sequencing, the proper band was isolated using gel extraction on a 0.5% agarose gel followed by purification with the E.Z.N.A.R gel extraction kit (Omega BioTek). The concentration of the DNA was measured using AccuClear™ (Biotium) following manufacturer protocols. The flow cell was loaded with 15 pM DNA with 5% vivoPhiX™. For NovaSeq™-based sequencing, samples were purified using PippinHT (Sage Science) and the concentration was measured via 4200 TapeStation (Agilent). The size selection, concentration measurement, and NovaSeq™ runs were performed by the University of Wisconsin Madison Biotechnology Center (UWBC).

Mapping Data Analysis: The FastQ output was merged using PEAR. A C++ script was used to filter poor-scoring reads based on Q-scores. Reads that passed the quality filter were then filtered on constant regions surrounding the barcode and TtgR variants. Barcodes that had read counts greater than 10 and were unique for a single TtgR variant were mapped to a that variant. If a barcode mapped to more than one TtgR variant, then the TtgR variant that had the most reads was selected if each other variant was less than 10% of the reads of the most abundant variant.

RNA-Seq preparation: RNA was harvested according to the RNA purification protocol. cDNA synthesis used approximately 3 μg total RNA, a primer encoding a 16 nt unique molecular identifier (UMI), and the Maxima™ H Minus Double-Stranded cDNA Synthesis Kit. The cDNA was purified using the DNA Clean & Concentrator™ 5 kit (Zymo). The Illumina® sequencing regions are added in 2 PCR reactions in the same manner as the MiSeq barcode-variant mapping reactions. Three sets of primers containing the Illumina® sequencing primer and a predefined barcode (ATCG, CGAT, and GTCA) were used in the first PCR reaction to add the Illumina® sequencing regions (11 cycles). One set of primers was used for each biological replicate. The first reaction is purified using the DNA Clean & Concentrator™ 5 kit (Zymo). The second reaction uses 4 μL of the first reaction and primers that add i5 and i7 indices in 8 cycles. The final amplicons are purified again. All replicates were combined in an equal molar ratio after purification.

Plasmids were harvested from the remaining culture of the RNA preparation step. The UMI was added to the plasmid-derived samples in a 2-cycle PCR reaction using 100 ng of template. The amplification of all DNA libraries followed an identical protocol to the RNA preparation.

The cDNA and DNA samples are sequenced using either a NovaSeq™ SP chip (test library) or a NovaSeq™ S4 chip (DMS and agnostic libraries) by the UWBC.

RNA-Seq Data Analysis: Fastq files were merged using NGmerge and filtered using Fastp based on average Q-score>Q30 for reads. Reads containing the 5′ and 3′ constant regions were isolated using UMITools and counted using Tally. Reads containing the central constant region were isolated and UMI sequences were removed with UMI-Tools. The barcodes were then counted with Tally. RNA-Seq barcodes were matched to mapped barcode-variant pairs with a Hamming distance tolerance of 1 using Seal (sourceforge.net/projects/bbmap/).

RNA-Seq barcodes that were successfully mapped to known barcode-variant pairs were analyzed across the induced RNA, induced DNA, control RNA, and control DNA samples. A barcode both had to be found in all four datasets to be included in downstream analysis. The read counts for a variant were then a sum of the barcode counts for all barcodes mapped and found in all four datasets. No read count threshold was imposed during analysis. The fold enrichment calculation uses equation (4).

$$Fold Enrichment = \frac{\frac{{RNA}_{+ Lig}}{{DNA}_{+ Lig}}}{\frac{{RNA}_{- Lig}}{{DNA}_{- Lig}}} \qquad ( 4 )$$

If biological replicates were available for each condition, the fold enrichment per variant was curated based on the coefficient of variation (CV). Percent deviation is calculated with equation (5).

$$CV = \frac{σ}{\overline{x}} \qquad ( 5 )$$

In this equation, σ is the standard deviation of the fold enrichment and X is the mean fold enrichment across replicates. A 30% CV cutoff was imposed for the agnostic dataset and a 20% deviation cutoff was imposed on the DMS dataset.

All variants were normalized to wildtype fold enrichment for each replicate. Heatmaps were constructed using the average performance of each variant after normalization.

Cell Sorting: An overnight culture is diluted 50× in phosphate buffered saline (137 mM NaCl, 2.7 mM KCl, 10 mM Na₂HPO₄, 1.8 mM KH₂PO₄) and placed on ice for 10 minutes prior to sorting. Sorting was performed on an SH800 (Sony) using the 488 nm laser and a 525±25 filter. Sorted cells were grown for 1 hour shaking at 37° C. in 5 mL LB. Kanamycin was added to a final concentration of 50 μg/mL and the culture was grown overnight. An aliquot of the sorted culture was stored at −80° C. in 25% glycerol. Plasmids were isolated from the remaining culture using the ZR Plasmid Miniprep™-Classic kit (Zymo).

Creating TtgR_pBBR1_SPS_V2: The plasmid containing the TtgR gene and the sfGFP gene under control of the TtgR operator sequence was created using two Gibson Assembly reactions. The sfGFP gene was under control of a modified TtgR operator sequence with canonical −10 (5′-TATAAT-3′) and −35 (5′-TTGACA-3′) elements in the promoter. The backbone contains the TtgR gene under an apFAB61-BBaJ61132 constitutive operator sequence, a kanamycin resistance marker, and the pBBR1 origin (TtgR_pBBR1). The sfGFP gene was inserted into the pBBR1 backbone following the standard methodology. Next, a terminator was placed at the 3′ end of the sfGFP gene according to protocol. This plasmid was labeled as TtgR_pBBR1_SPS_V2.

Creating TtgR_ColE1_SPS_V5: The pBBR1 origin was exchanged for a ColE1 origin. The sfGFP fragment was amplified from TtgR_pBBR1_V2 using primers specific for the sfGFP region with 5′ ends complementary to the destination ColE1 backbone and to the sfGFP amplicon. The TtgR gene was amplified from the TtgR_SC101BBa plasmid with primers containing complementary regions to the backbone and GFP amplicon. The sfColEl backbone amplicon contains a kanamycin marker and a ColE1 origin. Plasmids were labeled as TtgR_ColE1_SPS.

The sfGFP promoter was modified to have the wildtype TtgR operator sequence. sfGFP with the wildtype operator sequence was amplified from a separate plasmid using primers with overlap to the TtgR_ColE1_SPS plasmid. The backbone amplicon was amplified from TtgR_ColE1_SPS and consisted of the TtgR gene, the Kanamycin resistance marker, and the ColE1 origin. The plasmid was labeled as TtgR_ColE1_SPS_V2.

A third Gibson assembly reaction was required to insert stop codons and BsaI cut sites into the middle of the GFP gene to create the barcode insertion site. The stop codons and BsaI sites were encoded on overlapping primers and added to the TtgR_ColE1_SPS_V2 plasmid. The backbone as annealed to itself in a 1-part isothermal assembly. This construct was labeled TtgR_ColE1_SPS_V5.

Creating GFP control: To create a GFP positive control, the TtgR gene was removed from the TtgR_ColE1_SPS_V2 sfGFP gene. The sfGFP gene was amplified with primers complementary to the backbone. The BsaI cut sites and early stop codons were inserted into sfGFP in the same fashion as the creation of TtgR_ColE1_SPS_V5. The plasmid was labeled TtgR_ColE1_SPS_V3_GFPControl. Three pre-defined 20 nt barcodes (AAACCCTGTGCCAGAGGGTG (SEQ ID NO: 1), GAGTGACCTTAAGTCAGGGA (SEQ ID NO: 2), and GCTTCTGTCCAAGCAGGTTA (SEQ ID NO: 3)) were generated according to standard protocols. The barcodes were inserted into the TtgR_ColE1_SPS_V3_GFPControl using Golden Gate assembly.

OD600 optimization: mRNA levels were assayed at three different OD600 values: 0.6, 1.2, and approximately 2.8 (overnight growth). Testing was performed with the TtgR_ColE1_SPS_V2 plasmid with primers specific for the 5′ region of sfGFP. rrsA, a ribosomal subunit and constitutively expressed gene, was used as a reference. 2 mL cultures were grown and RNA harvested according to standard protocols. The abundance of the sfGFP and rrsA transcripts were measured via qRT-PCR following the standard protocol (data not shown)

Length Optimization: The OD600 0.6 induction samples were used to test the effect of different amplicon lengths on qRT-PCR fold enrichment. One of the TtgR_ColE1_SPS_V2 samples assayed with DMSO was used to calculate the primer efficiency of three different primer pairs. Each pair shared the same forward primer but had differing reverse primers that yielded amplicon lengths of 75 bp, 150 bp, and 300 bp. 0.001 ng, 0.01 ng, 0.1 ng, or Ing of RNA was added to Luna® Universal One-Step qRT-PCR mix (NEB) containing 4 μmol of each primer on ice. These RNA amounts were also assayed with the rrsA primers in the same manner. The abundance of the sfGFP and rrsA transcripts were measured via qRT-PCR following the standard protocol (data not shown).

Creating TtgR Test Library: The TtgR gene variants were isolated from a set of 16 pre-existing plasmids each containing a single TtgR variant. 100 ng of each amplicon was combined into a single aliquot and stored at −20° C. Barcodes for the RNA-Seq were 16 nt in length and were encoded on a ssDNA primer (IDT). The TtgR_ColE1_SPS_V5 backbone was amplified using primers that encompassed the sfGFP gene, the ColE1 origin, and the kanamycin resistance marker. The barcodes, TtgR gene variants, and backbone were assembled in a single Golden Gate reaction (NEB) according to standard protocols.

Mapping test library barcode-variant pairs: A 60 nt spacer was created to bring the random barcode and TtgR variants physically adjacent on the same plasmid to enable short read next generation sequencing mapping of barcode variant pairs. The test library plasmids were amplified with primers encoding BsaI cut sites that would place the spacer between the barcode and the TtgR variant region. The spacer was inserted into the backbone using Golden Gate (NEB) following standard protocols. The resulting library was sequenced on a 15M 2×250 MiSeq chip (Illumina®). Data analysis followed standard protocols.

RNA-Seq of test library: The induction of the test library used either 1 mM naringenin or DMSO as a control. DH10B containing each barcoded GFP Control plasmid were struck out on LB-kanamycin plates. One colony was selected from each barcoded DH10B and grown in 3 mL LB-kanamycin overnight. These barcoded control cultures were combined in equal ratio and added to the test library culture to a final composition of 0.25% control. The induced cultures were grown and prepared following standard protocols.

Validating RNA-Seq test library results: The test library frozen stock was struck out on LB-kanamycin and grown overnight. 16 colonies were selected and the TtgR variants were identified using colony PCR per standard protocols. 8 of the 16 total variants were verified via sequencing and were stored at −80° C. RNA was harvested from each variant in biological triplicate under 1 mM naringenin and DMSO conditions. qRT-PCR was used to assay barcode transcript enrichment.

Creating agnostic libraries: FuncLib-tolerated mutations were encoded into short oligos (Agilent) consisting of the TtgR gene region flanked by BsaI cut sites for Golden Gate assembly. Four pools of approximately 4,400 variants were created by randomly combining between 1 and 5 tolerated mutations. Each pool had unique priming sequences to isolate from a pooled sample. The pooled library was diluted to 0.005 μM in Tris-HCl (pH 7.5). Each pool was amplified using Kapa HiFi™ and 1 μL of the diluted library in 15 cycles in triplicate. The amplified reactions were pooled together and purified using the DNA Clean & Concentrator™ 5 kit (Zymo). The pooled oligos were cloned into the TtgR_ColE1_SPS_V5 backbone using Golden Gate assembly (NEB). The libraries with approximately 15 barcodes per variant, calculated by CFU/mL, were selected for RNA-Seq.

Mapping agnostic Libraries: The mapping process was performed as described in the general cloning methods. The spacer library was sequenced using an 2×250 NovaSeq SP chip (Illumina®) by the UWBC.

Agnostic RNA-Seq: The agnostic libraries were induced with the ligand (Table 2). DMSO, dH2O, and EtOH were included as solvent controls. The four pools were grown individually in 5 mL LB kanamycin overnight in triplicate. The four pools were combined prior to inoculation in 25 mL LB kanamycin for the RNA harvest. GFP Control barcoded cells were spiked into the combined agnostic replicates at a final concentration of 0.25%. The same pooled replicates were used for all ligand inductions. Read volumes were calculated by targeting 500 reads per barcode with the assumption that 50% of the reads will be lost due to filtering criteria.

TABLE 2
LIGANDS AND CONCENTRATIONS IN RNA-SEQ

| Ligand | Concentration mol/L |
|---|---|
| Tamoxifen | 0.00005 |
| Endoxifen | 0.00005 |
| 4-hydroxy tamoxifen | 0.00005 |
| N-desmethyl tamoxifen | 0.00005 |
| Naltrexone | 0.001 |
| Quinine | 0.0005 |
| Ellagic Acid | 0.00015 |
| Naringenin | 0.001 |
| Phloretin | 0.0003 |

RNA-Seq data analysis: Data analysis followed the RNA-Seq pipeline described above. Variants with data passing CV thresholds for more than 5 ligands and performed at least 1.5 times better than wildtype were selected for clustering. Missing data was imputed using KNN methods in SciKit Learn. The UPGMA algorithm with a correlation distance metric and a target of 12 clusters was used to cluster in SciPy. The number of clusters was selected by plotting the silhouette score against the number of clusters (data not shown). The relative positional entropy was calculated for each cluster compared to the total set of variants using equation (6):

$$RE = {\sum }_{a} {f}_{cluster , a} ( \frac{{f}_{cluster , a}}{{f}_{all , a}} ) \qquad ( 6 )$$

In this equation, a is the set of all amino acids observed at a single position and f is the frequency with which that amino acid is observed. This equation compares clustered sets of sequences compared to all possible agnostic sequences. This equation was only applied to clusters with more than 20 sequences.

Testing Top Hits: Top performing variants were selected based on the mean rank of each variant across the three biological replicates. These variants were encoded in gene fragments (Twist) and synthesized in a 96-well plate format. The fragments were resuspended to a final concentration of 10 ng/μL, pooled together, and cloned into the TtgR_ColE1_SPS_V2 backbone using Golden Gate Assembly. The resulting library was sorted based on fluorescence.

LB media was inoculated with 50 μL of the frozen stock of the library and grown overnight shaking at 37° C. Sorting was performed according to the Cell Sorting protocol. 500,000 cells were isolated of the lower 70% of the population based on fluorescence. Plasmids were isolated from the remaining culture using the ZR Plasmid Miniprep™-Classic kit (Zymo). DH10B (NEB) were transformed with the purified plasmid library according to the Library Creation protocol.

The abundance of variants was determined using next-generation sequencing. Sequencing amplicons were generated using primers that had complementarity to the TtgR gene around the gene fragment insertion site. The amplification process followed the “Barcode-variant mapping via next-generation sequencing” protocol. The concentration of the DNA was measured using Qubit™ Fluorometric Quantification (Thermo Fisher) following manufacturer protocols. The flow cell was loaded with 15 pM DNA with 5% vivoPhiX™. Sequencing was performed on a MiSeq instrument (Illumina®).

Fastq files were merged using NGmerge and filtered using Fastp based on average Q-score>Q30 for reads.

DMS Library synthesis: The TtgR DMS libraries were created from pre-existing TtgR DMS libraries. This DMS library was split into 6 different segments that encompassed the length of the TtgR gene. Each segment was a separate plasmid library. These segments were amplified separately and cloned into the TtgR_ColE1_SPS_V5 backbone using Golden Gate assembly (NEB). Libraries with approximately 10 barcodes per variant were selected for RNA-Seq.

Mapping DMS Libraries: The mapping process was performed as described in the general cloning methods. The spacer library was sequenced using an 2×250 NovaSeq SP chip (Illumina®) by the UWBC (data not shown).

DMS RNA-Seq: The DMS libraries were induced with either 50 μM tamoxifen, 50 μM endoxifen, or EtOH. The six pools were grown individually in 5 mL LB-kanamycin overnight in triplicate. Each segment was induced separately. Read volumes were calculated by targeting 500 reads per barcode with the assumption that 50% of the reads will be lost due to filtering criteria. Data analysis followed the RNA-Seq pipeline described above. The 90th percentile of positions by number of mutations outside the interquartile range of all variants were defined as functional hotspots.

Clonal validation of top hits: The top three unique variants for each ligand were identified based on the “Testing Top Hits” library. Each variant was encoded in short oligos (Twist) and cloned into the TtgR_ColE1_SPS_V2 backbone following general cloning methods. Colonies were selected and grown overnight consistent with general cloning methods. 3 μL overnight cultures were inoculated with 3 μL ligand such that the final concentration is consistent with Table 2 and LB-Kan was added to a final volume of 150 μL in a 96-well plate. Fluorescence was measured on a Synergy HTX (Biotek).

### Example 1: Validating RNA-Seq on a 16-Member Library

As illustrated in FIG. 2A, widespread adoption of transcription factor biosensors in biotechnology requires the ability to design known aTFs to bind to a diverse array of small molecules (FIG. 2A). To assay the function of a library of transcription factor variants, transcript quantity was assayed directly via RNA-Seq. One of the major challenges was linking the expression of a reporter gene to the transcription factor variant responsible for controlling its expression. Transcription factors can be uniquely identified using the expression of a short, randomized barcode as the reporter gene. To link the transcription factor to the barcode, a plasmid was created that contained both the transcription factor variant and the barcode on the same piece of DNA (FIG. 2B). A second construct was used to map the aTF variant to the barcode with next-generation sequencing (FIG. 2C). Once the transcription factor and barcode pairings are known, transcription factor function is a measure of the abundance of the barcodes during RNA-Seq (FIG. 2C). E. coli containing the plasmid library are dosed with either the target ligand or a vehicle control and harvested in log phase to obtain both total RNA and the library plasmids (FIG. 2D). The RNA provides a measure of function while the plasmids facilitate normalization to prevent library skew from affecting results.

A small test library of 16 TtgR variants that have differential response to naringenin was tested. Gene fragments encoding the variants were inserted with random barcodes into our expression vector. Barcodes were mapped to variants in a separate next-generation sequencing run. For barcodes that are mapped to multiple variants, the majority variant was selected if the read counts for each other variant amounted to less than 10% of the read count of the most abundant variant. Each variant in the test library had approximately 8,000 barcodes (FIG. 2E).

To analyze the performance of RNA-Seq on a transcription factor library, the RNA-Seq fold enrichment was compared to qRT-PCR fold enrichment. 8 of the 16 variants were isolated from random colony screening of the test library for individual quantification. The test library and clonal variants were dosed with either 1 mM naringenin or DMSO as a vehicle control. The RNA-Seq data was subset to barcodes that appeared in all four conditions (naringenin RNA, naringenin DNA, DMSO RNA, and DMSO DNA). The performance of a variant was calculated as the sum of the barcode counts for each variant (FIG. 2F). Comparison of the qRT-PCR fold enrichment and the RNA-Seq data showed high correlation (R2=0.83) (FIG. 2G). The RNA-Seq approach readily replicates differences observed via qRT-PCR across a range of functions.

A bigger library with 8,000 barcodes per variant will likely be too large to sequence thoroughly. Without being held to theory, it was hypothesized that down-sampling fewer barcodes per variant will affect the accuracy of the fold enrichment calculation. A Monte Carlo sampling approach was used to randomly select 10, 25, 50, and 100 barcodes per variant for 500 trials each and scored each sample by its correlation to the qRT-PCR dataset (FIG. 2H). Each bootstrap group shows, on average, similar correlation to the qRT-PCR assay compared to the 8,000 barcodes per variant. Thus, larger libraries can be accommodated with a smaller barcode to variant ratio to reduce the sequencing volume requirements.

### Example 2: Identifying Novel Sensors in the Agnostic Library

An agnostic library against a range of small molecules was created to find new biosensors (FIGS. 6A-I). Four derivatives of tamoxifen (Tam), a breast cancer therapeutic, were selected to create specific and multi-specific sensors. Tamoxifen is a selective estrogen receptor (ER) modulator that is metabolized by cytochrome P450 into 4-hydroxy-tamoxifen (4Hy) and N-desmethyltamoxifen (Ndes). These two metabolites are then catabolized to endoxifen (End). Endoxifen and 4Hy are the most abundant metabolites and show high activity. By using sensing platforms for active metabolites of Tam like End and 4Hy, physicians can ensure maximum efficacy during treatment.

Quinine (Quin), naltrexone (NaI), and ellagic acid (EllA) were also selected as targets for the agnostic library. Quinine is a small molecule therapeutic used to treat malaria. It is isolated from the bark of the cinchona tree; a sensor for quinine will be useful for creating a biosynthetic pathway in prokaryotes for scalable production. Furthermore, a quinine sensor may be useful for monitoring quinine resistance and variations in pharmacokinetics during treatment. Naltrexone is used to treat addiction as an opioid receptor antagonist. Chemically, it shares many similarities to other compounds that interact with the opioid receptors like morphine and heroin. Naltrexone is chemically distinct from TtgR's native ligands and thus poses a challenging target for affinity engineering. By obtaining an opioid sensor, we can develop portable devices for quick detection of this class of compounds. Ellagic acid is a plant polyphenol with a highly conjugated chemical structure and shares chemical features with native ligands of TtgR (FIG. 6).

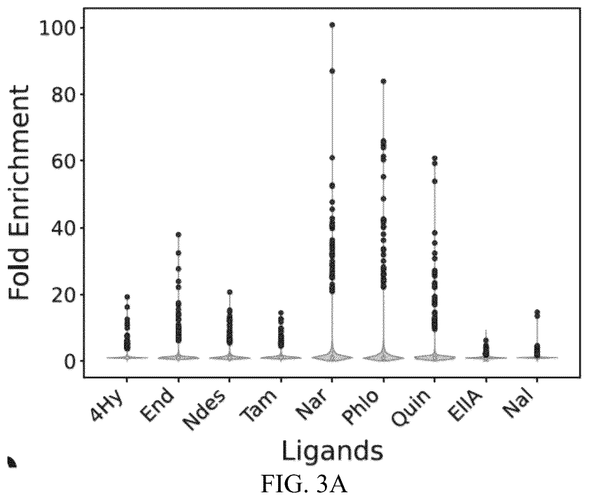

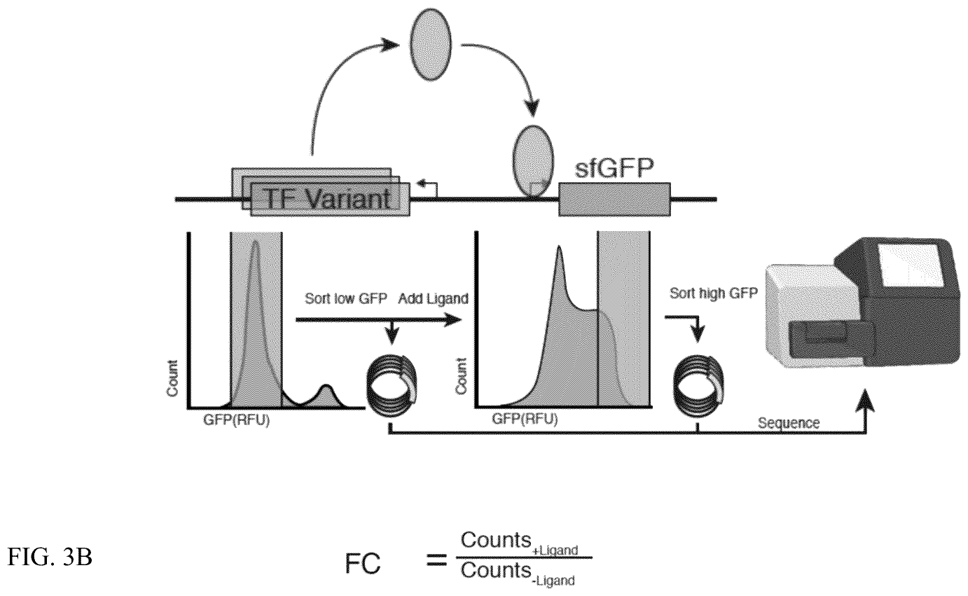

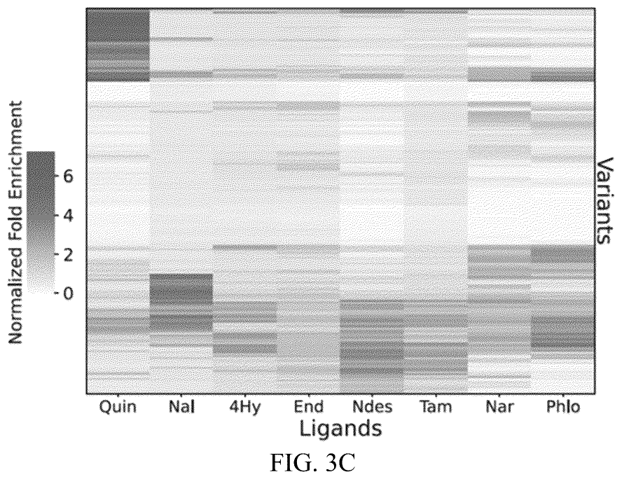

A set of computationally stable substitutions at key positions in the ligand binding domain of TtgR was obtained using the FuncLib tool and a 17,737-member library was constructed comprising of 1-4 mutations. Mapping barcode-variant pairs identified 17,533 variants (98.8%) with an average of 20 barcodes per variant (data not shown). RNA-Seq of the 16N barcodes corresponding to TtgR variants contained barcodes associated with 17,365 TtgR variants (97.9%). Each ligand had a wide range of functional responses; top performing variants are candidates for novel biosensors (FIG. 3A).

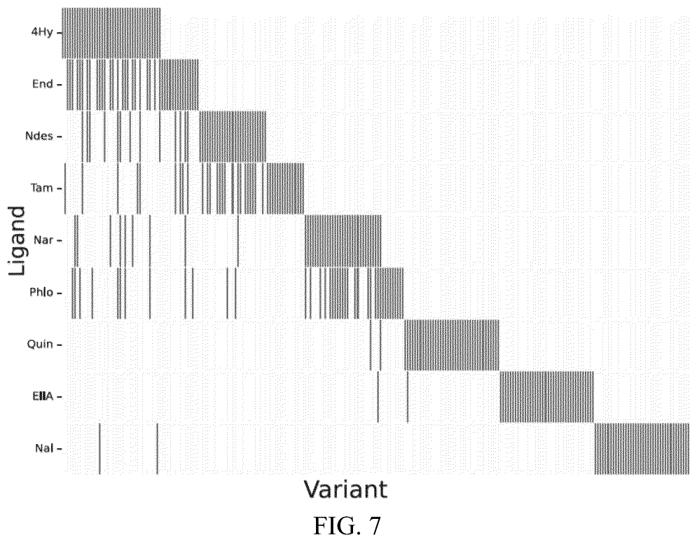

The variants in the top standard deviation for any one ligand were selected for further analysis (points) (FIG. 3A). These variants performed the best against naringenin and phloretin, the native ligands of TtgR, and had the weakest response to ellagic acid. The top 40 sequences for each ligand were selected for validation in a fluorescence-based assay. The best performers of the naringenin and phloretin ligands shared many variants (FIG. 7). Similarly, the tamoxifen, endoxifen, 4-hydroxytamoxifen, and N-desmethyltamoxifen sets shared variants (FIG. 7) The quinine, naltrexone, and ellagic acid top performing variants were unique to each ligand (FIG. 7).

The 251 unique top variants were cloned into an expression vector containing sfGFP under control of the TtgR operator sequence (FIG. 3B). Variants capable of repressing transcription in the absence of any small molecule were first isolated via cell sorting. These variants were then exposed to each ligand and the high fluorescence cells were sorted (FIGS. 8A and B). The performance of these variants is the fold change in percent population of the high fluorescence and repressed sorts.

The fold change of variant abundance in the sorted library indicated that each ligand had a subset of functional transcription factors. However, the library showed no change in fluorescence when inoculated with ellagic acid, suggesting that a fluorescence assay is insufficient for screening this ligand. Variants showed response to all other ligands (FIG. 3C). Although the naltrexone top variants only showed high activity for naltrexone, these variants showed generalized response across most ligands (FIG. 3C). In contrast, quinine top performers are generally specific for quinine (FIG. 3C). Surprisingly, naringenin and phloretin top variants do not show strong response to these ligands, but the low fold change may be due to the large number of variants that were isolated in the high fluorescence sort during the sorting process (data not shown). The percent abundance in the induced state will thus differ less for each variant compared to the abundance in the repressed state. The cross reactivity of the top performing variants to eight of the nine ligands highlights the broad applicability of this approach. The majority of sequences with high function in the RNA-Seq dataset also show response in a fluorescence-based assay.

The fold change of variant abundance in the sorted library indicated that each ligand had a subset of functional transcription factors. However, the library showed no change in fluorescence when inoculated with ellagic acid, suggesting that a fluorescence assay is insufficient for screening this ligand. Variants showed response to all other ligands (FIG. 3C). Although the naltrexone top variants only showed high activity for naltrexone, these variants showed generalized response across most ligands (FIG. 3C). In contrast, quinine top performers are generally specific for quinine (FIG. 3C). Surprisingly, naringenin and phloretin top variants do not show strong response to these ligands, but the low fold change may be due to the large number of variants that were isolated in the high fluorescence sort during the sorting process (data not shown). The percent abundance in the induced state will thus differ less for each variant compared to the abundance in the repressed state. The cross reactivity of the top performing variants to eight of the nine ligands highlights the broad applicability of this approach. The majority of sequences with high function in the RNA-Seq dataset also show response in a fluorescence-based assay.

The top three unique variants for each ligand were individually generated (See methods). Variants often showed high fold induction to more than one ligand. Certain variants, such as those with high response to naltrexone and quinine, had low response to other ligands. Similarly to the library screen, the tamoxifen, endoxifen, 4-hydroxytamoxifen, and N-desmethyltamoxifen variants had cross-reactivity to the other tamoxifen metabolites. The highest performing naringenin and phloretin variants were also found to respond to the tamoxifen derivatives.

The top three unique variants for each ligand were individually generated (See methods). Variants often showed high fold induction to more than one ligand. Certain variants, such as those with high response to naltrexone and quinine, had low response to other ligands. Similarly to the library screen, the tamoxifen, endoxifen, 4-hydroxytamoxifen, and N-desmethyltamoxifen variants had cross-reactivity to the other tamoxifen metabolites. The highest performing naringenin and phloretin variants were also found to respond to the tamoxifen derivatives.

The top three unique variants for each ligand were individually generated (See methods). Variants often showed high fold induction to more than one ligand. Certain variants, such as those with high response to naltrexone and quinine, had low response to other ligands. Similarly to the library screen, the tamoxifen, endoxifen, 4-hydroxytamoxifen, and N-desmethyltamoxifen variants had cross-reactivity to the other tamoxifen metabolites. The highest performing naringenin and phloretin variants were also found to respond to the tamoxifen derivatives.

### Example 3: Analysis of Library Data

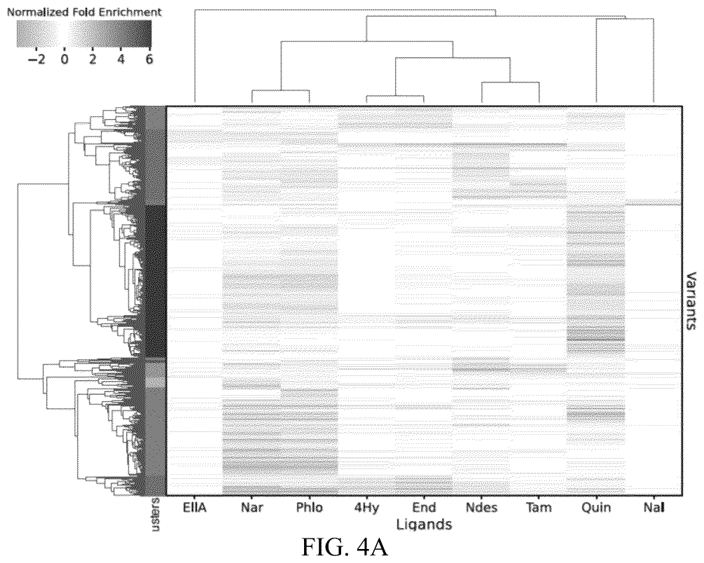

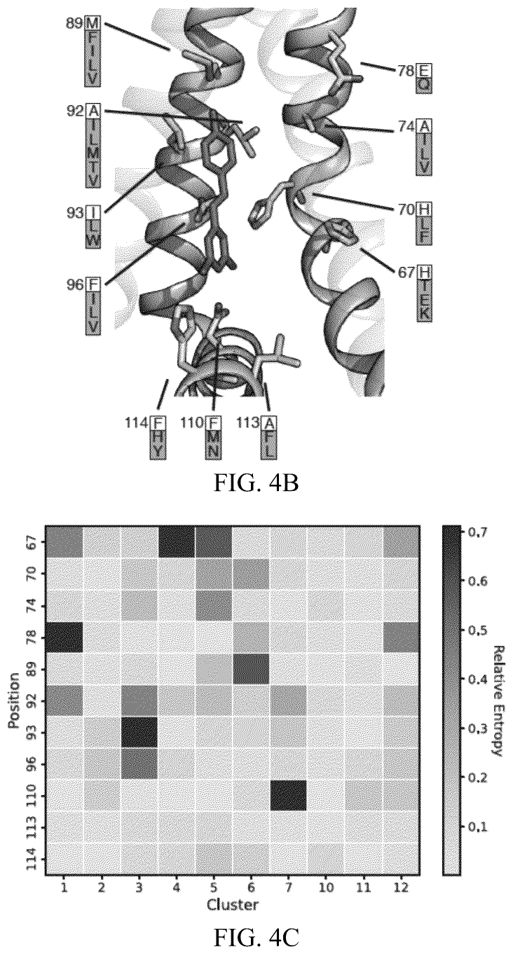

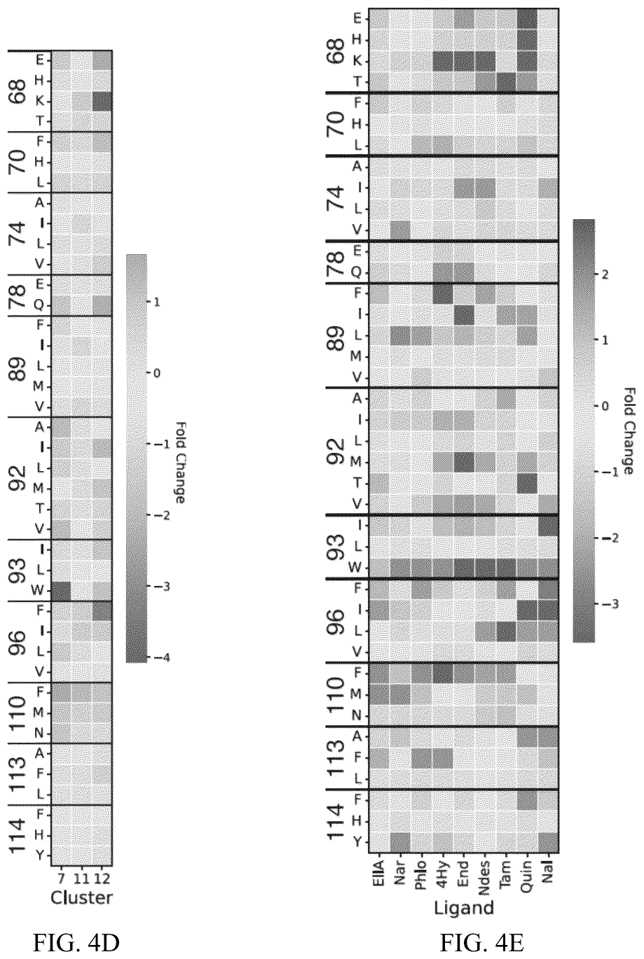

The agnostic library was designed to provide a set of stable substitutions without optimizing affinity for any ligand. This approach enables the same library to be screened across multiple ligands, as each aTF variant has the potential to interact with a new subset of small molecules due to the redesigned ligand binding pocket. Given these design constraints, it is expected that many variants will have similar ligand specificity profiles. The RNA-Seq can be leveraged to gain information about mutations that create and affect function across all aTF variants and ligands. All 16,190 variants (91.2% of all variants) with data for at least 6 of the 9 ligands were selected and the missing data was imputed using KNN imputation (see methods). Variants that performed at least 1.5 fold better than wildtype (3,135 variants) on at least one of the nine ligands were selected for hierarchical clustering via the UPGMA algorithm with a correlation distance metric and a target of 12 clusters (FIG. 4A, data not shown). The ligand clusters (top dendrogram) are grouped appropriately based on the chemical structure (FIG. 6A-I). The tamoxifen ligands are most closely related by their performance. Similarly, naringenin and phloretin, the two native ligands of TtgR, also cluster together. In contrast, the ligands with the most structural diversity (ellagic acid, naltrexone, and quinine) are the most distant.

The variant clusters (FIG. 4A, left dendrogram) display unique sequence specificity profiles based on the normalized fold enrichment across the 9 ligands. Cluster 1 (top) is characterized by higher 4-hydroxytamoxifen and endoxifen normalized fold enrichment. The third cluster contains variants with high naltrexone response. The fourth cluster is primarily composed of variants with high quinine normalized fold enrichment. Cluster 7 is characterized by variants with high N-desmethyltamoxifen and tamoxifen normalized fold enrichment.

The sequence profiles of the cluster members were characterized to understand the important substitutions that contribute to the unique specificity profile of each cluster. The agnostic library is a combination of selected mutations across a limited number of positions in the binding pocket with potential to directly interact with small molecules (FIG. 4B). The relative positional entropy of all mutable positions for each cluster in comparison to the entire 16,190 sequences was calculated (see methods). Relative positional entropy quantifies the change in amino acid distribution after clustering. In this context, high relative entropy indicates that certain amino acids are preferred at a particular position once clustered. Each cluster except cluster 2, cluster 10, and cluster 11 has one or more positions with high selective pressure (FIG. 4C). L113 and H114 show low relative positional entropy across all clusters, suggesting that these two positions do not contribute to unique protein-ligand interactions (FIG. 4C). These two residues are located at the bottom of the binding pocket and have the potential to make hydrogen bond and nonpolar interactions with small molecules that are oriented in the native “vertical” binding pose for many ligands in wildtype TtgR (FIG. 4B). Surprisingly, N110 is adjacent to L113 and H114 and has the potential to make similar interactions with ligands at the bottom of the binding pocket as L113 and H114, but has high entropy in Cluster 7 (FIG. 4B, 4C). N-desmethyltamoxifen and tamoxifen thus interact with this position in preference over L113 and H114. All other positions make up one face of the ligand binding pocket and have at least one cluster that has high relative positional entropy.

Comparing amino acid sequence preferences at high selectivity positions across clustered sequences and top performing variants for each ligand can identify distinct amino acid trends for each ligand. Some clusters are very similar to one another across the nine ligands. Clusters 7, 11, and 12 all share high naringenin and phloretin fold response but differing tamoxifen response profiles (FIG. 4A). Cluster 7 has higher N-desmethyltamoxifen and tamoxifen response while cluster 12 has higher 4-hydroxytamoxifen and endoxifen response (FIG. 4A). Cluster 11 shows high naringenin and phloretin response but does not respond to any of the tamoxifen ligands. The positions in clusters 7, 11, and 12 in the top 80th percentile of relative positional entropy were examined to identify large changes in amino acid distribution before and after clustering. Cluster 7 has two positions with large changes: 92 and 110. Cluster 11 has no positions with high relative entropy. Cluster 12 shows high relative positional entropy at 67, 78, and 92. In Cluster 7, position 92 favors the wildtype leucine substitution over all possible mutations. Only the best tamoxifen variants also show this behavior (FIG. 4D). N-desmethyltamoxifen, tamoxifen, and phloretin favor the valine substitution while naringenin does not. In contrast, naringenin favors alanine, isoleucine, or methionine at 92 (FIG. 4D). These differences highlight the generalizable approach of the agnostic library.

Selecting amino acid substitutions from a limited set at a limited number of positions can drastically change ligand response. Cluster 11, which contains variants with high response naringenin, phloretin, and quinine, contains no positions with high relative entropy. Since naringenin and phloretin are two of the native ligands of TtgR and every position has a high percentage of wildtype residues due to the limited number of mutations in the library, many variants show little change in the amino acid distributions in response to clustering (FIG. 4E). The small change in amino acid distribution before and after clustering across the positions implies that wildtype response is largely maintained regardless of amino acid substitution. Often, the change in amino acid frequency in the clusters are matched by the changes observed in the top performers of associated ligands. For example, cluster 7, which has high N-desmethyltamoxifen and tamoxifen shows similar amino acid preferences as the top variants of N-desmethyltamoxifen (FIG. 4D, 4E). The amino acid preferences of each cluster and the top performing variants imply that the sequence-based analysis can highlight key functional substitutions that are associated with high ligand response.

The agnostic design scheme of the aTF library creates a set of variants with the potential to interact with a wide variety of ligands while having a constrained set of mutations that are selected for stability. By clustering the variants based on their performance across ligands, we can gain insight into the amino acid preferences at each position and an initial understanding of the importance of each in conferring novel ligand affinity. Comparing the sequence profiles of clusters with shared naringenin and phloretin performance highlights the unique solutions that can give rise to novel patterns of ligand specificity. The library thus contains variants with unique sets of ligand specificity, creating a wide variety of potential sensors with tunable response profiles.

### Example 4: Dms of Ttgr Against Endoxifen and Tamoxifen Highlights Functional Hotspots

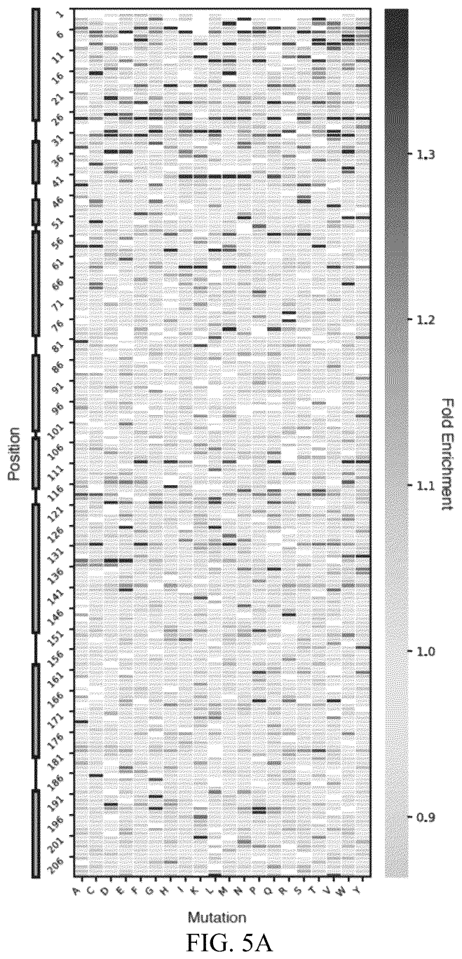

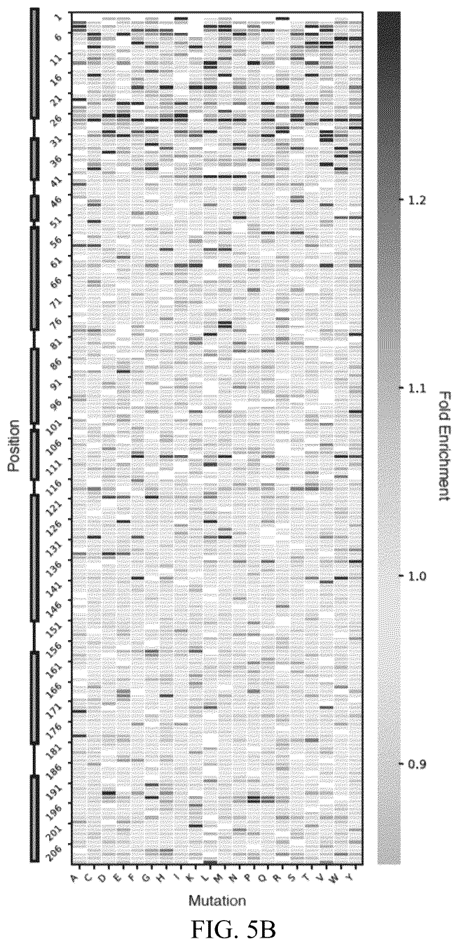

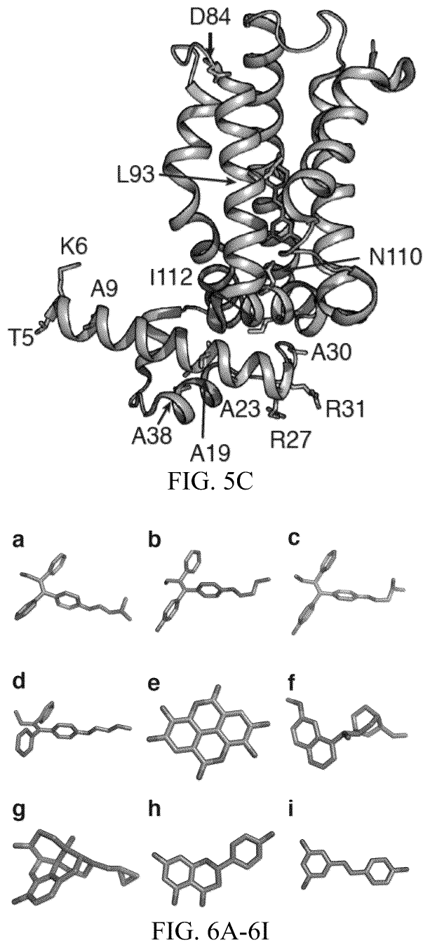

One benefit of an RNA-Seq based screening workflow is that many libraries can be screened in parallel to create a functional landscape of variants. A deep-mutational scan library of TtgR consisting of single point mutations of every position to all 19 other amino acids against tamoxifen and endoxifen was tested. The library was split into 6 different pools spanning amino acids 1-39, 40-77, 78-115, 116-153, 154-191, and 192-210. The majority of single point mutations have little effect on function, with 3,897 variants in the endoxifen dataset and 3,996 variants in the tamoxifen dataset between 1.2 and 0.8 of wildtype fold enrichment (FIG. 5A, 5B).

The DNA binding domain has regions of high and low function across multiple substitutions. The helix of the HTH motif that directly interacts with the major groove of the operator sequence contains many positions where the majority (61%) of mutations decrease aTF function (FIG. 5A, 5B). Substitutions at positions between 81 and 101 are often detrimental (approximately 53%) to protein function than the wildtype residue. These positions correspond to a solvent-facing region of a helix in the ligand binding pocket, suggesting an allosteric role in gene expression control. In contrast, 65% of mutations to positions between 116 and 153 confer increased function (FIG. 5A, 5B). These positions compose a single helix of the ligand binding pocket, but high-performing positions are agnostic of orientation on the helix. Surprisingly, the helix associated with dimerization (186-210) also contains many positions where the majority of substitutions (approximately 64%) are beneficial to protein function.

The variants that show high performance or low performance may be indicative of hotspots that are critical for protein design. Hotspots were selected based on the number of mutations whose performance fell outside the interquartile range of fold enrichment normalized to wildtype performance (see methods). The majority of identified hotspots are near the DNA binding domain and the interface between the DNA binding domain and the ligand binding domain.

Positions within the DNA binding domain can be classified as solvent interactions, DNA interactions, or potential allosteric interactions (FIG. 5C). T5, K6, A9, and R27 are located in the first helix of the DNA binding domain and do not have any significant interactions with the operator sequence, the ligand, or the other TtgR monomer. The high mutability of positions 5, 6, 9, and 27 may be because these positions have nonspecific interactions with the solvent. A38 is located in the recognition helix that interacts with the major groove of the operator sequence and has a direct effect on the capacity of TtgR to repress gene expression. Six of the seven positions at A38 drastically reduce function. A19, A23, A30, and R31 have possible structural or allosteric functions based on their location. A19 and A23 make van der Waals interactions with 137 and L40 in the recognition helix and possibly stabilize the position of the recognition helix conformation. These positions may also be important for the allosteric changes that occur in response to ligand binding that decrease affinity for the operator sequence. The backbone amide of A30 makes a hydrogen bond with D118. Mutating A30 to polar residues increases function by creating additional interactions with A30 and R31 on the opposite monomer. R31 makes hydrogen bond interactions with T120 and D122 on the opposite monomer. The hydrogen bond interactions are substituted for van der Waals interactions in the majority of mutations at this position.

The positions in the ligand binding domain either directly interact with the ligand or interface with the DNA binding domain. L93 and N110 directly interact with the ligand and are mutable positions in the agnostic library. Mutating position 93 results in a loss of function for the majority of mutations. However, tamoxifen and endoxifen yield different sets of mutations that improve function. In contrast, mutations at position 110 largely improve function with the exception of the cysteine mutation, which is consistent across both ligands. I112 is located at the interface of the ligand binding domain and the DNA binding domain. The side group of 112 is located between F24 and Y25 in the DNA binding domain. The size and polarity of this position is important as only the leucine substitution increases function. Any substitution to residues with different shape or polarity results in a loss of function. D84 is a solvent-accessible position located in a loop in the ligand binding pocket. Selecting positions that have multiple substitutions that confer either high or low function via the RNA-Seq approach identifies functional hotspots in TtgR in the DNA binding domain and the ligand binding domain. The importance of these hotspots can be rationalized based on their location in the structure of TtgR.

### EXAMPLE 5: Improvements to the SensorSeq Method

Initially, functional scores for each aTF variant were computed by summing the observed counts for each barcode sequence and calculating the log ratio of RNA and DNA sequencing reads for each variant. These ratios are computed for factors assayed with and without condition, and the difference between these scores indicates the magnitude effect of the ligand on gene expression. Processing scores with this method led to a low average R2 correlation between biological replicates of 0.54 (FIG. 9A). To address this issue, the presence of individual barcode measurements was leveraged, treating each barcode measurement for a specific variant as a technical replicate. By applying estimation with restricted maximum likelihood (RML) to combine these technical replicates, followed by another round of RML to merge biological replicates, a significant improvement in the average correlation between biological replicates to an R2 of 0.93 was achieved (FIG. 9A). Scores calculated with this enhanced method also show a higher correlation to Fluorescence-based validation metrics, improving the average correlation across conditions from a spearman correlation of 0.62 to 0.87 (FIG. 9B). This enhancement will substantially reduce false positive and negative rates, thereby increasing the value of SensorSeq data for replication and utilization in machine learning algorithms.

As part of the RMLE optimization process, a confidence scoring mechanism was developed for the functional scores of each biosensor. These confidence scores, determined during RMLE, integrate information from sequencing counts for each barcode, variability among technical replicates, and variability among biological replicates. Analysis reveals that all screened ligands exhibit varying confidence levels for each variant measurement (FIG. 9C). This metric serves as a valuable filter for selecting biosensors for clonal validation by streamlining the validation process for high-sensitivity biosensors through improved consistency between high-throughput SensorSeq assays and clonal validation assays.

Initial SensorSeq experiments were conducted in 25 mL flasks, limiting the number of ligands that could be screened in a single day and increasing costs for hard-to-procure ligands. However, repeating the assay for one of the initial ligands in a 2 mL culture demonstrated high correlation (Spearman correlation 0.92) of fitness scores across experiments (FIG. 9D). Scaling down reaction volumes not only reduces screening costs for expensive ligands but also enables high-throughput screening in deep 96-well plates. This increased throughput capacity facilitates the assessment of novel functions and specificities against hundreds of molecules within a single day.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of selecting allosteric biosensor proteins which bind a target ligand, comprising
providing a library of replicating plasmids, each plasmid comprising an expression construct and primer binding sites for next generation sequencing of at least a portion of an allosteric protein variant or an allosteric domain variant and a reporter, wherein each expression construct comprises
a gene encoding the allosteric protein variant or the allosteric domain variant which is operably linked to a first promoter for expression of the allosteric protein variant or allosteric domain variant, and
functionally linked to the gene, is the reporter comprising a barcode sequence for identification of the allosteric protein variant or allosteric domain variant, wherein the reporter is operably linked to a second promoter,
wherein, when the target ligand binds expressed allosteric protein variant or allosteric domain variant, expression of the reporter from the second promoter is activated, and when the target ligand does not bind expressed allosteric protein variant or allosteric domain variant, expression of the reporter from the second promoter is inactivated, or
wherein, when the target ligand binds expressed allosteric protein variant or allosteric domain variant, expression of the reporter from the second promoter is inactivated, and when the target ligand does not bind expressed allosteric protein variant or allosteric domain variant, expression of the reporter from the second promoter is activated;
mapping each allosteric protein variant or allosteric domain variant in the library to the barcode sequence or sequences associated with the allosteric protein variant or allosteric domain variant and assigning variant-barcode pairs;
growing a population of cells transfected with the library of replicating plasmids in the presence of the target ligand and isolating target ligand total RNA and target ligand library plasmids;
performing next generation sequencing to determine a quantity of each barcode in the target ligand total RNA;
from the quantity of each barcode and the assigned variant-barcode pairs determining a fold enrichment for each allosteric protein variant or allosteric domain variant in the target ligand total RNA, wherein the fold enrichment for each allosteric protein variant or allosteric domain variant in the target ligand total RNA is normalized by the target ligand library plasmid; and either
(i) from the quantity of each barcode and the assigned variant-barcode pairs determining a fold enrichment for each allosteric protein variant or allosteric domain variant in the target ligand total RNA, wherein the fold enrichment for each allosteric protein variant or allosteric domain variant in the target ligand total RNA is normalized by the target ligand library plasmid; and selecting a subpopulation of variants with the highest fold enrichment as the selected allosteric biosensors, or
(ii) treating each barcode for a specific variant as a technical replicate, applying estimation with restricted maximum likelihood (RML) to combine the technical replicates, performing a second round of RML to merge biological replicates; and selecting a subpopulation of merged biological replicates as the selected allosteric biosensors.

2. The method of claim 1, wherein the allosteric protein or allosteric domain comprises a DNA binding domain that, in the presence of the target ligand, either inactivates or activates expression of the reporter from the second promoter.

3. The method of claim 2, wherein the allosteric protein or allosteric domain comprises an allosteric transcription factor.

4. The method of claim 3, wherein the allosteric transcription factor comprises such as TetR, LacI, TtgR, MphR, AraC, or LysR.

5. The method of claim 1, wherein the allosteric protein or allosteric domain associates, directly or indirectly, with a DNA binding domain that, in the presence of the target ligand, either inactivates or activates expression of the reporter from the second promoter.

6. The method of claim 5, wherein the allosteric protein or allosteric domain and the DNA binding domain comprise a PhoP/PhoQ, EnvZ/OmpR, KdpE/KdpD, and ComA/ComP.

7. The method of claim 1, wherein the first promoter is a constitutively active promoter.

8. The method of claim 1, wherein the barcodes have a length of 16 to 24 nucleotides.

9. The method of claim 1, wherein each allosteric protein variant or allosteric domain variant is associated with 10 to 100 barcodes.

10. The method of claim 1, wherein the library comprises 5,000 to 20,000 allosteric protein variants or allosteric domain variants.

11. The method of claim 1, wherein mapping comprises direct sequencing of the expression constructs, or removing a constant region of the expression constructs of the library of plasmids between the variable region of the allosteric protein variant or allosteric domain variant and the barcodes, and ligating the variable region to the barcode sequence, and performing high throughput sequencing to assign the variant-barcode pairs.

12. The method of claim 1, wherein selecting the subpopulation of variants with the highest fold enrichment as the allosteric biosensors comprises selecting 10 to 100 variants.

13. The method of claim 1, wherein the reporter further comprises a coding sequence for a detectable marker protein operably linked to the second promoter.

14. The method of claim 13, further comprising normalizing the fold enrichment for each allosteric protein variant or allosteric domain variant using a method comprising
providing control replicating plasmids comprising a control reporter comprising a barcode sequence for identification of the allosteric protein variant or allosteric domain variants, wherein the control reporter is under control of the second promoter,
growing a population of cells transfected with the control replicating plasmids,
performing next generation sequencing to determine a quantity of each barcode in the control total RNA, and
normalizing the fold enrichment for each allosteric protein variant or allosteric domain variant by the average fold enrichment of each control barcode in the total RNA.

15. The method of claim 13, further comprising validating the subpopulation selected allosteric biosensors by determining expression of the detectable marker protein in the presence and absence of the target ligand.

16. The method of claim 1, further comprising determining a functional score for each of the selected allosteric biosensors using confidence scoring.

17. The method of claim 1, wherein the target ligand is a biological molecule, an environmental molecule, a drug, a metal ion, a carcinogenic molecule, a food contaminant, or an environmental contaminant.

18. The method of claim 1, wherein selecting allosteric biosensor proteins is done in a high throughput assay.

19. A device comprising a substrate, cell or chamber comprising the allosteric biosensor selected by the method of claim 1.

20. The device of claim 19, wherein the device is suitable for use in high throughput screening, single cell analysis, online monitoring, evolution, or dynamic pathway evolution, cell-free biosensing, whole-cell biosensing, control of cellular functions, or inducible promoters for gene expression control.


SEQUENCE LISTING (3 sequences)

SEQ ID NO: 1 - DNA, 20 bp, synthetic construct
  source 1..20: mol_type = other DNA
aaaccctgtgccagagggtg

SEQ ID NO: 2 - DNA, 20 bp, synthetic construct
  source 1..20: mol_type = other DNA
gagtgaccttaagtcaggga

SEQ ID NO: 3 - DNA, 20 bp, synthetic construct
  source 1..20: mol_type = other DNA
gcttctgtccaagcaggtta